(19) United States
(12) Patent Application Publication
(10) Pub. No.: US 2005/0209556 A1
(43) Pub. Date: Sep. 22, 2005

(54) Implantable intravascular delivery device

(75) Inventors: Patrick A. Tresco, Sandy, UT (US); Mayank D. Patel, San Diego, CA (US)

Correspondence Address: PROCOPIO, CORY, HARGREAVES & SAVITCH LLP, 530 B STREET, SUITE 2100, SAN DIEGO, CA, 92101, US

(73) Assignee: MICROISLET, INC.

(21) Appl. No.: 10/804,818

(22) Filed: Mar. 19, 2004

(51) Int. Cl. A61M 31/00

(52) U.S. Cl. 604/93.01

(57) ABSTRACT

A device and method are described for the delivery of cells, tissues, enzymes and/or pharmacological agents for the treatment or prevention of diseases, disorders or deficiencies. The device is placed intravascularly and includes a chamber that houses living cells delimited by a membrane on either side that physically separates the cells from the blood stream and the central lumen of the catheter. The device can be inserted over a guidewire and permits flushing and reloading of the central lumen with viability supporting factors that sustain the cells in the outer chamber for long indwelling times without removing it from the body. In addition, the central lumen can be used to deliver therapeutic substances or withdraw blood. The new intravascular catheter can be used for the treatment or prevention of a variety of diseases and disorders, that may use the implantation of living cells, tissues, enzymes or pharmacological agents or for use, for example, for non-therapeutic purposes that may involve sustained intravascular release of biological factors as, for example, in stimulating growth of farm animals to augment the production of meat.

### FIELD OF THE INVENTION

The present invention relates to a device for implantation into the vasculature of a host for delivery of therapeutic agents or therapeutic agents produced from cells within the device. Included in the invention are methods of using the device for the treatment or prevention of a variety of diseases and disorders.

### BACKGROUND OF THE INVENTION

Various methods, membranes, devices and methods of their manufacture have been proposed and evaluated as a means of transplanting cells and their secreted products into the human body and are collectively referred to in the patent literature as bioartificial implants. They all share a common principle of operation, that is, the cells are sequestered inside a chamber bounded by a semipermeable membrane. Long term cell viability relies on the sustained diffusive exchange of nutrients and waste products with adjacent vascularized tissue. (U.S. Pat. Nos. 6,372,244, 6,113,938, 6,322,804, 4,911,717).

The membrane of all types of bioartificial implants must allow sufficient transport of chemical agents to keep the encapsulated cells alive and responsive to a changing external microenvironment. Availability of key nutrients and diffusive transport across and throughout the device is critical to the successful implementation of this technology.

Sufficient diffusive exchange requires proximity to capillaries and involves transport from the blood in the capillary, across the capillary wall, across the extracellular space and into the device. The performance of such devices described to date are further compromised by the fact that they become encapsulated upon implantation by a well recognized foreign body response that further hinders diffusion and compromises the cell carrying capacity of the device. Delayed neovascularization of the device, on the one hand, and fibrotic encapsulation inhibiting transport between the device and the host are factors complicating the efficacy of these devices.

There are three major types of devices described in the scientific and patent literature for implantation of cells into various tissue compartments including: Planar disk designs, (U.S. Pat. No. 6,372,244, retrievable bioartificial implant having dimensions allowing rapid diffusion of oxygen and rapid biological response to physiological change, processes for their manufacture and methods for their use; U.S. Pat. No. 5,855,613 retrievable bioartificial implants having dimensions allowing rapid diffusion of oxygen and rapid biological response to physiological change); Hollow fiber-based designs (U.S. Pat. Nos. 6,322,804 and 6,083,523 for implantable biocompatible immunoisolatory vehicle for the delivery of selected therapeutic products); and Geometric solid-based designs (U.S. Pat. No. 5,964,804 close vascularization implant material; U.S. Pat. No. 5,916,554 use of pouch for implantation of living cells; U.S. Pat. No. 6,511,473 implantable bioartificial active secretion system; and U.S. Pat. No. 6,485,723 enhanced submucosal tissue graft constructs).

Besides their mode of operation, each of the aforementioned designs was developed for placement within a body cavity, such as the abdominal cavity adjacent to the alimentary canal and its associated peritoneal tissue, or for placement into a subcutaneous site.

Newer designs include a means of removing and replacing the cells within the chamber or include a mechanism for retrieving the entire device. As previously described, to trigger a healing response, bioartificial devices rely on a neovascularization response following device implantation, that is, the development of new blood vessels immediately adjacent to the cell-containing membrane. The implanted device being encapsulated within a layer of well-vascularized fibrotic tissue that not only impedes device performance, but becomes damaged during the surgical procedure needed to remove or retrieve the device resulting in local hemorrhage and tissue damage.

All such bioartificial implants have proven unsatisfactory for reasons including one or more of the reasons set forth above.

The insertion of catheters into blood vessels to facilitate the injection or the removal of fluids or to maintain a passageway in an unobstructed condition is well known. In this respect, vascular catheters are used to deliver therapeutic substances by infusion under pressure directly into a blood vessel. Those skilled in the art will recognize a plethora of applications [Grossman's Cardiac Catheterization, Angiography, and Intervention Donald S. Baim (Editor), William, M.D. Grossman (Editor) Lippincott Williams & Wilkins Publishers; 6th edition (September 2000); Broaddus W C, Gillies G T, Kucharczyk J. Minimally invasive procedures. Advances in image-guided delivery of drug and cell therapies into the central nervous system. Neuroimaging Clin N Am. 2001 Nov; 11(4): 727-35].

Gaskill U.S. Pat No. 4,911,717 describes an intravascular artificial organ consisting of a double lumen catheter comprised of a continuous length of semipermeable membrane with convective filtration MWCO specified at 50,000 Daltons connected to a subcutaneous port that allows flushing and reloading of the device without removing it from the body.

To date, there has been no catheter as defined above that can be used for delivering cells and their released products into the bloodstream, or for modification of the microenvironment of the bloodstream due to the enzymatic activity of the cells or other agents immobilized or released from the catheter, while also delivering or withdrawing fluids from the bloodstream.

### SUMMARY OF THE INVENTION

The intravascular device of the present invention is adapted to provide for the delivery of cells, tissues, enzymes and/or pharmacological agents to an individual for the treatment or prevention of diseases, disorders or deficiencies.

The device comprises an elongated, flexible, tubular body with a hematologically compatible outer membrane surface. The tubular body has proximal and distal ends. A chamber that runs lengthwise houses living cells, tissues, enzymes, or other long-term sustained release system, whether liquid, gel or solid.

The chamber is delimited by the outer membrane or jacket, which separates the chamber from the blood stream; and the casing of a conduit which is disposed within the chamber between the proximal and distal ends.

The conduit serves for insertion over a guidewire. In some embodiments, the conduit is attached to a catheter and an access port through which the conduit can be flushed and reloaded with a viability supporting solution that sustains the cells in the outer chamber for long indwelling times without removing it from the vasculature. The conduit also provides access to the bloodstream, facilitating the injection or withdrawal of fluids. In another aspect the conduit can be filled with contrast agent for device placement.

One or a plurality of supporting members are situated in the chamber, disposed for engaging an interior surface of the outer membrane. The supporting member provides mechanical support, prevents kinking, facilitates distribution of the cells within the outer chamber, and permits retrieval without loss of cells.

In another aspect, the invention provides methods of treating an individual in need of therapeutic treatment which requires administration of a biological moiety. The method involves a step of introducing the device of the invention into the central venous vasculature for a sufficient period to deliver a sufficient amount of said biological moiety to the individual to achieve a therapeutic effect.

In yet another aspect of therapeutic treatment, the device is implanted into the individual's peritoneal cavity or into subcutaneous tissue.

### DESCRIPTION OF DRAWINGS

The features, objects and advantages of the invention will become apparent to those skilled in the art from the following detailed illustrations of preferred embodiments, especially when considered with the accompanying drawings, indicated by the following legends.

FIG. 1 shows an embodiment of the intravascular cell containing catheter with access port to the central lumen, a syringe connected to the access port, the catheter located in a peripheral vein and extending toward the vena cava;

FIG. 2 shows an embodiment of the intravascular cell containing catheter disposed over a guidewire and located in a peripheral vein and extending toward the vena cava.

FIG. 3 is a perspective view of the intravascular catheter, the casing cut away to expose the central lumen, outer chamber, supporting member, and cells or cell clusters in the outer chamber.

FIG. 4 is a side sectional view of the intravascular catheter.

FIG. 5 is a side sectional view of the intravascular catheter in which pores are situated in the casing of the central lumen.

FIG. 6 is a cross sectional view taken along line 6-6 of FIG. 4

FIG. 7 shows cross-sectional views diagramatically illustrating diffusion of solutes, including biological moieties, within the device and between the device and the bloodstream.

### DESCRIPTION OF THE INVENTION

Unless otherwise defined, all technical and scientific terms have the same meaning as commonly understood by one of ordinary skill in the art to which this invention belongs. All publications, patent applications, patents and other reference material mentioned are incorporated by the reference. In addition, the materials, methods and examples are only illustrative and are not intended to be limiting.

General Description and Definitions

The practice of the present invention will employ, unless otherwise indicated, conventional techniques within the skill of the art in (1) culturing cells and tissues, (2) drug delivery (3) biomaterials, (4) biochemistry; (5) molecular biology, (6) artificial organs (7) tissue engineering. Such techniques are explained fully in the literature. [See, e.g. Culture of Animal Cells: A Manual of Basic Technique, 4th edition, 2000, R. Ian Freshney, Wiley Liss Publishing; Animal Cell Culture, eds. J. W. Pollard and John M. Walker; Neural Cell Culture: A Practical Approach, vol. 163, ed. James Cohen and Graham Wilkin; Maniatis et al., Molecular Cloning: A Laboratory Manual; Molecular Biology of The Cell, Bruce Alberts, et.al., 4th edition, 2002, Garland Science; Pharmaceutical Biotechnology, eds. Daan J. A. Crommelin and Robert D. Sindelar, 1997, Harwood Academic Publishers). Principles of Tissue Engineering, 1^{st} edition, eds. Lanza et al., 1997, R. G. Lander Company; Engineered Materials Handbook, Desk edition, 1995, ASM International; Engineered Materials Handbook Vol 3: Adhesives and Sealants, 1990, ASM International; Plastics Extrusion Technology Handbook, 2n edition, S Levy and J F Carley, 1989 Industrial Press Inc.;. Relevant periodicals include Cell Tissue Research; Cell; Science; Nature; Science, Biomaterials, Journal of Biomedical Materiasl Research, journal of Membrane Science, Artificial Organs, Tissue Engineering.]

### DEFINITIONS

The following terminology will be used in accordance with the definitions set out below in describing the present invention.

A “biologically active moiety” is a tissue, cell, or other substance, which is capable of exerting a biologically useful effect upon the body of an individual in whom a vehicle of the present invention containing a biologically active moiety is implanted. Thus, the term “biologically active moiety” encompasses cells or tissues which secrete or release a biologically active molecule, product or solute; cells or tissues which provide a metabolic capability or function, such as the removal of specific solutes from the bloodstream; or a biologically active molecule or substance such as an enzyme, trophic factor, hormone, or biological response modifier.

The present invention applies to the administration of beneficial agents in general, which include any physiologically or pharmacologically active substance. The beneficial agent may be any of the agents which are known to be delivered to the body of a human or an animal such as drug agents, medicaments, vitamins, nutrients, or the like.

Drug agents which may be delivered by the present invention include drugs which act on the peripheral nerves, adrenergic receptors, cholinergic receptors, the skeletal muscles, the cardiovascular system, smooth muscles, the blood circulatory system, synaptic sites, neuroeffector junctional sites, endocrine and hormone systems, the immunological system, the reproductive system, the skeletal system, autacoid systems, the alimentary and excretory systems, the histamine system, and the central nervous system. Suitable agents may be selected from, for example, proteins, enzymes, hormones, polynucleotides, nucleoproteins, polysaccharides, glycoproteins, lipoproteins, polypeptides, steroids, analgesics, local anesthetics, antibiotic agents, anti-inflammatory agents, corticosteroids, ocular drugs, antibodies and synthetic analogs of these species.

Biocompatible Materials: As used herein, the term “biocompatible” refers collectively to both the intact vehicle and its contents. Specifically, it refers to the capability of the implanted intact vehicle and its contents to avoid detrimental effects of the body's various protective systems and remain functional for periods of days to weeks In addition to the avoidance of protective responses from the immune system, or foreign body fibrotic response, “biocompatible” also implies that no specific undesirable cytotoxic or systemic effects are caused by the vehicle and its contents such as would interfere with the desired functioning of the vehicle or its contents.

A “blood compatible” material is one that will not induce adverse reactions in the patient or put the patient at risk of undesirable reactions as a result of contact with blood, such as health jeopardizing thrombosis.

The term “individual” refers to a human or an animal subject, that is, a host for the device

The term “implant,” as used herein, is defined to include all living tissues, cells, and biologically active substances intended to be implanted into the body of an individual, as well as the act of implanting or transferring these tissues and cells into an individual. These tissues and cells include, without limitation, tissue and cells removed from a donor animal, tissue and cells obtained by incubation or cultivation of donor tissues and cells, cells obtained from viable cell lines, cells obtained from stem cells; cells obtained by genetic engineering of primary tissue, cell lines or stem cells, biologically active products of cells and tissues, pharmaceuticals, drugs, enzymes, eutrophics, antibodies and the like. Tissues may perform a useful biological function either by secreting a therapeutic or trophic substance or by removing a toxic or harmful one. An example of the latter would be removal of various fatty substances from serum to reduce blood lipid levels.

As defined in the Oxford Dictionary of Biochemistry and Molecular Biology (Oxford University Press, 1997), the term “culture” refers to 1(a) a collection of cells, tissue fragments, or an organ that is growing or being kept alive in or on a nutrient medium (i.e. culture medium); (b) any culture medium to which such living material has been added, whether or not it is still alive. 2. the practice or process of making, growing, or maintaining such a culture. 3. to grow, maintain or produce a culture.

A “cell” is the basic structural unit of all living organisms, and comprises a small, usually microscopic, discrete mass of organelle-containing cytoplasm bounded externally by a membrane and/or cell wall. Eukaryotes are cells, which contain a cell nucleus enclosed in a nuclear membrane. Prokaryotes are cells in which the genomic DNA is not enclosed by a nuclear membrane within the cells.

“Culture medium” refers to any nutrient medium that is designed to support the growth or maintenance of a culture. Culture media are typically prepared artificially and designed for a specific type of cell, tissue, or organ.

Unless otherwise specified, the term “cells” means cells in any form, including but not limited to cells retained in tissue, cell clusters, and individually isolated cells.

“Tissue culture” refers to 1. the technique or process of growing or maintaining tissue cells (cell culture), whole organs (organ culture) or parts of an organ, from an animal or plant, in artificial conditions; 2. any living material grown or maintained by such a technique.

“Tissue” refers to any collection of cells that is organized to perform one or more specific function.

The term “diffusion” refers to the spontaneous mixing of one substance with another when in contact or separated by a permeable membrane or microporous barrier. The rate of diffusion is proportional to the concentration of the substances, i.e. solutes and solvents, and increases with temperature. The theoretical principles are stated in Fick's laws.

The term “diffusive flux” means the velocity by which a substance moves from one point to another resulting form ambient kinetic energy derived from the immediate environment in the absence of pressure gradients [Diffusion—Mass Transfer in Fluid Systems, E. L. Cussler. ISBN 0-521-29846-6. Cambridge University Press.]

The term “molecular weight cutoff” (MWCO) refers to the point or limit at which 90% of a solute is rejected from transport across a semipermeable barrier driven by a convective force or pressure gradient It is a term used to describe the filtering or separation capability of a membrane where a fluid is flowing through a membrane driven by a pressure gradient.

The term “jacket” as used herein refers to the outer membrane of the device

The term “delivery port” refers to a specialized chamber attached to the proximal portion of a catheter located outside the blood vessel usually located subcutaneously with a rubber wall on one side that faces the skin and can be accessed with a needle for the purposes of injecting medications, solutions or used to withdraw blood.

The term “conduit” refers to refers to the inner lumen, which is surrounded by a casing that may or may not be permeable depending on the preferred embodiment of the present invention.

The term “catheter” refers to a flexible tubular device that is implanted into a blood vessel for the purposes of infusing fluids or withdrawing blood

### THE DEVICE

The device of the invention is a bioartificial implant which is a biocompatible device suitable for short-or long-term implantation into the central venous vasculature. The device comprises elements of a bioartificial implant and an intravascular catheter for delivery of therapeutic moieties directly into the blood stream.

The inventions set forth herein provide an intravascular bioartificial implantable device 10 which also maintains an opening in a blood vessel 15, and methods of using the device for sustained modification of the extracellular fluid microenvironment of mammals and through which fluids can be injected or removed.

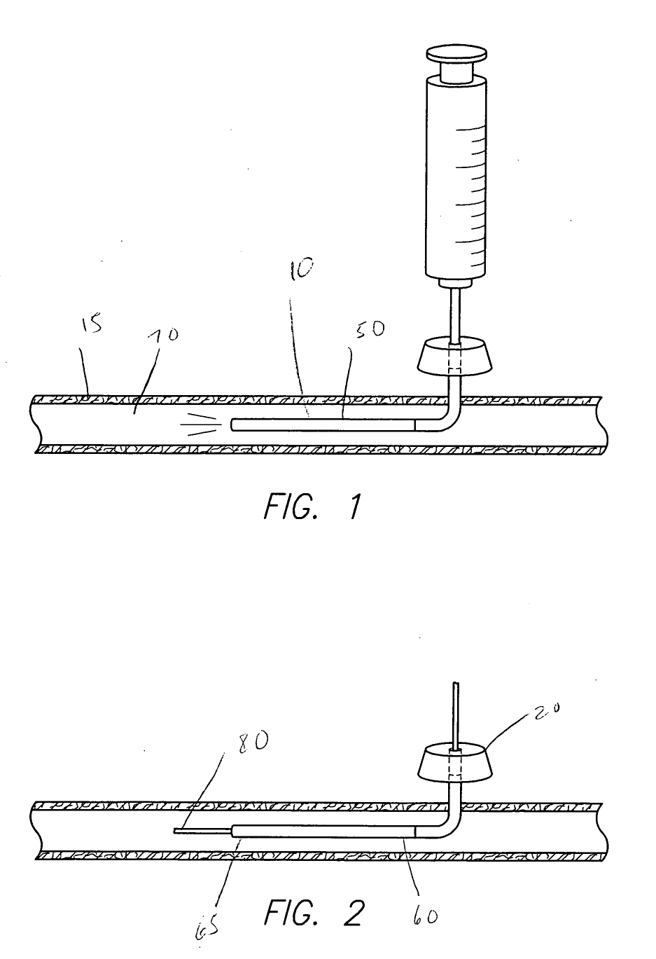

Referring to FIG. 1, there is shown a perspective view of the device 10 used to deliver cells, tissues or enzymes and/or pharmacological agents for the treatment or prevention of diseases, disorders or deficiencies.

In one aspect, the device is comprised of a delivery port 20, which in FIG. 1 is an implantable access port having a reservoir with at least one inner opening communicating with the lumen 25 of the device. The reservoir of limited volume is sealed at its proximal end by and is accessible through a septum used for infusion of viability sustaining factors intended to support the viability of cells in the chamber 30 of the device. The port 20 can be made of any number of materials known to those skilled in the art such as plastic and metals or composites including but not limited to polyurethane, silicone, stainless steel, polycarbonate and polysulfone. The port is connected to the conduit 35 within the tubular body by a catheter 40, in fluid communication between the access port and the proximal end of the lumen of the conduit through a flexible tethering section 45.

One version of delivery ports is well known in the art as implantable treatment devices, which often serve as injection ports for providing a treatment material, such as a drug in fluid form, directly to the vascular system of a mammal. A number of such devices are known (e.g. U.S. Pat. No. 4,673,394, incorporated by reference), and may be broadly described as having a housing that defines a reservoir for holding the treatment material, and a catheter leading from the housing for interconnecting the reservoir with the vascular system. Existing implantable devices are adapted to be sutured into place within the body. One type has a catheter permanently affixed to the reservoir housing. The second type has a catheter, which fits over a male tube projecting from the housing, and is secured thereto by placing an external collar about the catheter. This device permits the housing to be sutured in position, and the catheter to be installed in a vein and sized before connection of the catheter to the housing. In any case, the delivery or access port 20 provides a fluid outlet or inlet, which establishes secure fluid communication between the port 20, the catheter 40, and the lumen 25 of the device.

The tubular body, which in preferred embodiments is flexible, is bound at least in part by a jacket 50 or outer wall, which is a permeable membrane. A preferable version of this jacket has an outer surface which is a hematologically compatible material, and is sufficiently permeable to maintain viability of the biological moieties 55 housed in a chamber 30 described next. The tubular body has proximal 60 and distal 65 ends and includes a chamber 30 along its length for housing biological moieties. The jacket 50 is a permeable membrane that physically separates biological moieties disposed in the chamber from the blood stream 70. The casing 75 of the conduit 35 separates the chamber 30 from the lumen 25 of the device. The casing also can be permeable.

Substances and methods of applying them to membranes and other medical devices to achieve hemocompatible, surfaces are well known in the art (Carmeda™ North America, San Antonio, Tex.). These materials provide passive or inert surfaces for thermoplastics, rubbers, metals, wovens, and filter media that are anti-thrombotic, as well as anti-inflammatory and anti-infective, and are especially useful for central venous catheters.

FIG. 2 shows the conduit sized for over-the-wire insertion having an open distal end 65 that allows insertion over a guidewire 80 and permits flushing and reloading of the lumen with medium. In one embodiment, the medium is a viability supporting nutrient solution, which is disposed in the lumen, the lumen being permeable to the nutrients. The nutrient medium sustains the cells disposed in the chamber 30 for long indwelling times without removing the device from the vasculature.

The lumen can be used to facilitate the injection or withdrawal of fluids or can be filled with contrast agent for device placement.

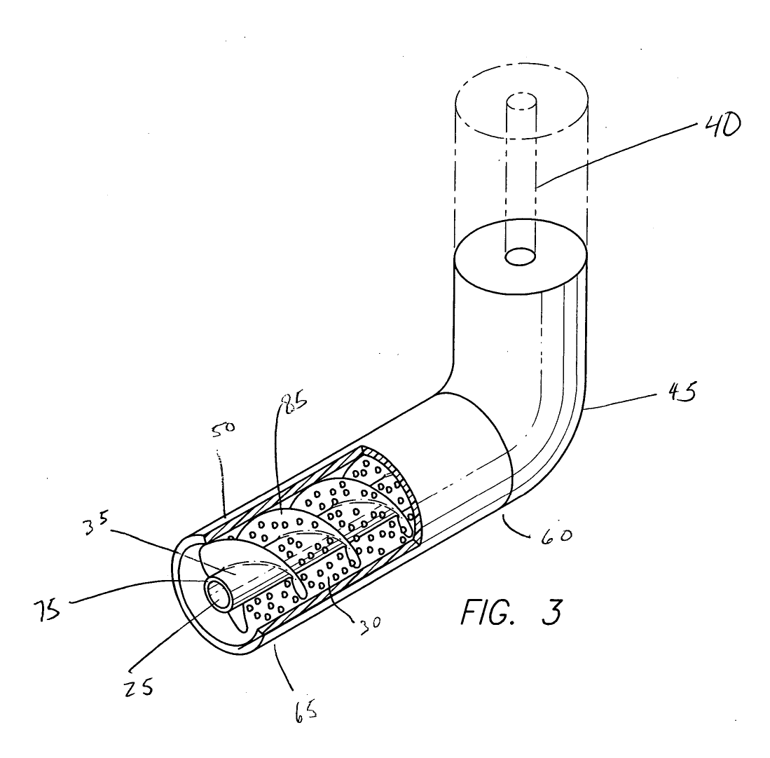

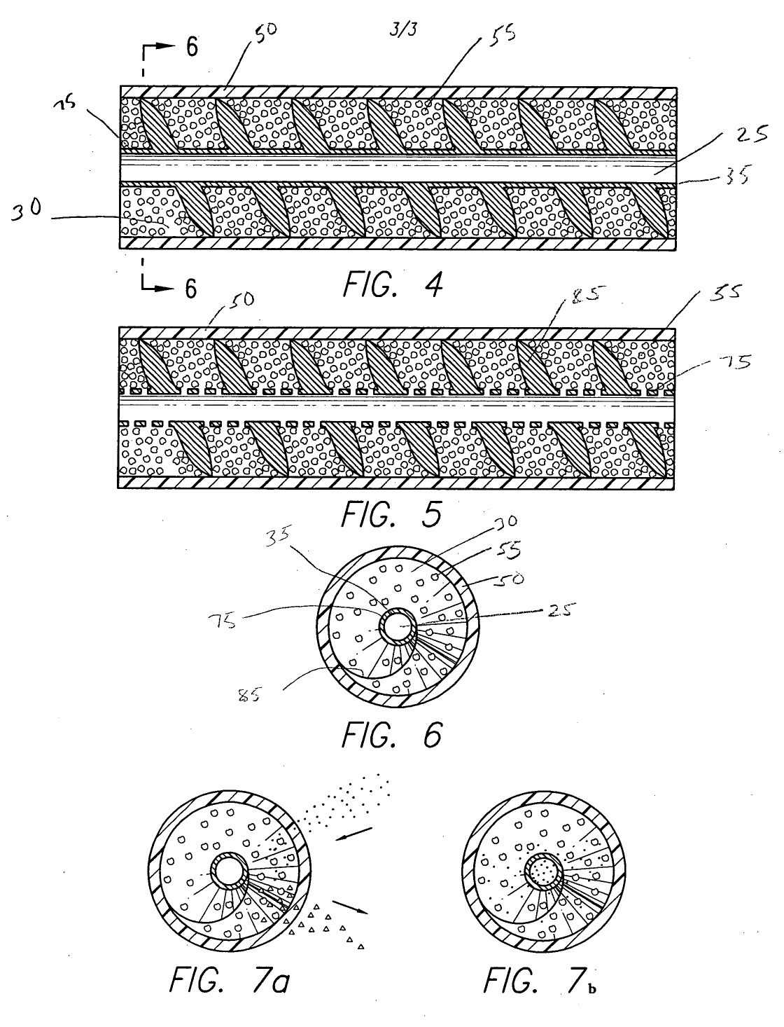

FIGS. 3, 4, 5 show support member 85 within the chamber 30, disposed against the inner surface of the jacket, which provides mechanical support, prevents kinking, and facilitates distribution of biological moieties within the chamber, and permits retrieval of the device without loss of the biological moieties.

Materials which may be used for the supporting member 85 should be sufficiently strong to ensure that the outer membrane jacket will not break, under stresses to which the device would be subjected during implantation or retrieval or under stresses due to the pressures generated during operation. The supporting member may be formed of chemically inert and biocompatible, natural or synthetic materials or thermoplastics which are known in the art. The material of the supporting member is preferably a non-bioerodible material such as polypropylene or an alloy of titanium or nitinol.

Preferred materials are those acceptable for human implants. In general, materials suitable for use in the supporting member of the outer jacket are elastomeric or flexible materials including the non-reactive polymers listed above, as well as elastomers in general, such as silicone rubbers, and polyurethanes. Typical materials of construction suitable for the supporting member according to the present invention include non-reactive thermoplastic polymers or biocompatible metals or alloys. The polymers include acrylonitrile polymers such as acrylonitrile-butadiene-styrene terpolymer, and the like; halogenated polymers such as polytetraflouroethylene, polychlorotrifluoroethylene, copolymer tetrafluoroethylene and hexafluoropropylene; polyimide; polysulfone; polycarbonate; polyethylene; polypropylene; polyvinylchloride-acrylic copolymer; polycarbonate-acrylonitrile-butadiene-styrene; polystyrene; and the like. Metallic materials useful for a supporting member include stainless steel, titanium, platinum, tantalum, gold, and their alloys, as well as gold-plated ferrous alloys, platinum-plated ferrous alloys, cobalt-chromium alloys, titanium nitride coated stainless steel and nitinol and its alloys.

Intravascular Catheters

Although distinguished by their structure, intravascular catheters and the device of the present invention deliver agents to an individual and can be used to withdraw blood. The use of intravascular catheters is well known in the art. Since the mid 1990s, peripherally inserted central catheters (PICCs), also known as indwelling central venous catheters (CVCs), have become an important means of central venous access, frequently used to deliver outpatient courses of intravenous therapy. Tunneled devices are used for long indwelling times, which have complication rates reported as low as 0.2% and have been known to those that practice the art to be used for periods up to three years.

It will be understood that variations in various dimensions of the device 10 are desirable to accommodate or take into account variations in patient anatomy, age and disease state as described below.( see explemplary dimensions).

Accordingly the outer chamber of the present invention can be made to have different volumes of biological moieties so that the devices can deliver over longer or shorter periods larger doses of biologically effective molecules having greater or lesser efficacy.

FIG. 7 shows how various biological moieties can be used or combined with a sustained release system located within the inner tube lumen or contained in the outer chamber. For example, FIG. 7a shows a drug or biologic (triangles) released from the chamber (outward arrow) of the device and diffusing into the blood stream. The inward arrow of FIG. 7b signifies nutrients from blood stream diffusing into the chamber. In other instances (FIG. 7b), a drug or biologic or nutrient factor diffuses from the lumen into the chamber through the permeable casing. In some instances, solutes or moieties which originate from the lumen may diffuse through the chamber, through the jacket into the bloodstream as a means of delivering agents (i.e. biologically active moieties or solutes) to the bloodstream.

FIG. 1 shows a syringe attached to the access port, exemplifying that the implant device can also function as an intravascular catheter for infusing drugs or biologics into the bloodstream or withdraw blood without interfering or disturbing or otherwise mechanically perturbing the contents of the chamber.

Exemplary Dimensions of the Device

The typical device in an adult patient is 50-60 cm in length and sizes are tailored for variation s in patient and arm vein anatomy. The present device can be placed in any one of a number upper arm veins and extend into the superior vena cava. Pediatric or young patients vary from 2-4 french (1 french equals approx. 0.33 mm), while most adult patients will receive 4-6 french dual lumen catheters. For adult applications, the device can be as short as 2 cm. Preferably it is in a range of 10 cm to as long as about 60 cm (or longer for larger dimensioned subjects). For pediatric applications, the device is preferably configured in shorter lengths depending on the size of the subject.

Biocompatibility of the Device

Because of its biocompatibility, the device is suitable for long-term isolation of biologically useful cells and/or substances.

The primary function of the outer jacket of the device is to physically isolate the biological moieties or sustained deliver system so that it may be retrieved to terminate therapy and completely remove the catheter and its contents. In some cases, the outer jacket may be immunoisolatory. That is, it protects biological moieties, in particular, cells disposed in the chamber of the device from the immune system of the individual in whom the device is implanted. It does so (1) by preventing harmful substances of the individual's body from entering the chamber core of the device, (2) by minimizing contact between the individual and inflammatory, antigenic, or otherwise harmful materials which may be present in the chamber and (3) by providing a spatial and physical barrier sufficient to prevent immunological contact between the isolated moiety and portions of the individual's immune system.

The device can be any generally elongate tubular configuration suitable for implantation into the vasculature and appropriate for maintaining therapeutic or biological activity of the biological moieties within the chamber, and providing access for delivery of the product or function to the bloodstream of the host.

The device must provide, in at least one dimension, sufficiently close proximity of any isolated cells in the chamber to the surrounding bloodstream in the vasculature of the individual to maintain the viability and function of the isolated cells. However, the diffusional limitations of the materials used to form the device do not in all cases solely prescribe its configurational limits. Certain additives can be used which alter or enhance the biological needs of the enclosed biological moieties of the basic device. For example, the internal medium can be supplemented with oxygen-saturated perfluorocarbons, thus reducing the needs for immediate contact with blood-borne oxygen. This will allow isolated cells or tissues to remain viable while agents are released from the device into the surrounding bloodstream. References and methods for use of perfluorocarbons are given by Faithful, N. S. Anaesthesia, 42, pp. 234-242 (1987) and NASA Tech Briefs MSC-21480, U.S. Govt. Printing Office, Washington, D.C. 20402, incorporated herein by reference.

Alternatively for clonal cell lines such as PC12 cells, genetically engineered hemoglobin sequences may be introduced into the cell lines to produce superior oxygen storage or hemoglobin or like polymers may be distributed in the outer chamber as a storage depot to maintain the viability of the biological moiety. NPO-17517 NASA Tech Briefs, 15, p. 54.

Accordingly, the present invention provides an osmotic and diffusion controlled implantable delivery device, and more particularly, a delivery system with an outer permeable jacket which physically isolates the chamber contents from being dispersed in the individuals blood stream. Rapid termination of therapy is achieved by removing the entire device from the patient

The Jacket—Outer Permeable Jacket (OPJ)

The outer permeable jacket (OPJ) portion of the device is constructed of a biologically inert polymer manufactured in a manner which makes the material permeable with a diffusive flux that exceeds 1×10-6 cm/sec for molecules below 100 kDa. The general properties of these membranes are discussed in Broadhead, K, Biran R, and P A Tresco, Hollow fiber membrane diffusive permeability regulates encapsulated cell line biomass, proliferation and small molecule release, Biomaterials, 2002 (24):4689-99.

The outer jacket allows passage of diffusible substances, but prevents the release of larger particles like living cells from entering into the bloodstream, at rates that would be biologically or medically significant. More specifically, the jacket is produced in such a manner that it has transport characteristics that are determined in part by the type and extent of immunological concerns based on the type of biological moiety to be delivered

In specialized cases where the biological moiety may be immunologically incompatible, the outer membrane transport properties may be tailored as known to those skilled in the art of making immunoisolatory bioartificial implant systems and, in the case of such non immunoreactive biological moieties as stem cells and autografts where rejection is not a problem, the outer jacket is freely permeable.

Thus the outer jacket surrounding the chamber is permeable, biocompatible, and in certain cases, may be immunoisolatory. It is produced in such a manner that it is free of isolated cells, and surrounds (i.e., isolates) the chamber, thereby preventing contact between any cells in the chamber and the recipient's body, or otherwise avoiding an unintended response between the host and the device.

That is, it does not elicit a detrimental host response sufficient to result in rejection of the implanted device or to render it inoperable. Neither does the jacket elicit unfavorable tissue responses such as fibrosis or create life endangering emboli.

First, the materials used to form the device are substances selected based upon their ability to be compatible with, and accepted by, the tissues of the recipient of the implanted device. Substances are used which are not harmful to the recipient or to the isolated biologically active moiety. Preferred substances include polyurethanes and blends thereof or permeable silicone based elastomers. Particularly preferred substances are those which are moderately hydrophobic, i.e. those having a solubility parameter as defined in Brandrup J., et al. Polymer Handbook 3rd Ed., John Wiley & Sons, N.Y. (1989), between 8 and 15, or more preferably, between 9 and 14 (Joules/m.sup.3).sup.1/2. The polymer substances are chosen to have a solubility parameter low enough so that they are soluble in organic solvents and still high enough so that they will partition to form a proper membrane. Such polymer substances should be substantially free of labile nucleophilic moieties and be highly resistant to oxidants and enzymes even in the absence of stabilizing agents. The period of residence in vivo, which is contemplated for the particular device must also be considered: substances must be chosen which are adequately stable when exposed to physiological conditions and stresses. There are many materials which are sufficiently stable, even for extended periods of residence in vivo, such as periods in excess of one or two years.

The jacket of the device can optionally include substances which decrease or deter the inflammatory response, and/or generate or foster a suitable local environment for the implanted cells or tissues. For example antibodies to one or more mediators of the immune response could be included. Available potentially useful antibodies such as antibodies to the lymphokines such as tumor necrosis factor (TNF), and to interferons (IFN) can be included in the matrix precursor solution. Similarly, an anti-inflammatory steroid can be included. Christenson, L., et al., J. Biomed. Mat. Res., 23, pp. 705-718 (1989); Christenson, L., Ph.D. thesis, Brown University, 1989, incorporated by reference.

In cases when the jacket is immunoisolatory, it protects cells in the chamber of the device from the immune system of the individual in whom the device is implanted. It does so (1) by preventing harmful substances of the individual's body from entering the core of the device, (2) by minimizing contact between the individual and inflammatory, antigenic, or otherwise harmful materials which may be present in the chamber and (3) by providing a spatial and physical barrier sufficient to prevent immunological contact between the isolated moiety and detrimental portions of the individual's immune system.

The thickness of this physical barrier can vary, but it will always be sufficiently thick to prevent direct contact between the biological moieties on either side of the barrier. The thickness of this region generally ranges between 5 and 200 microns; thicknesses of 10 to 100 microns are preferred, and thickness of 20 to 50 microns are particularly preferred.

Using the device, it is possible to deliver high molecular weight products or to provide metabolic functions pertaining to high molecular weight substances, or products having a wide range of molecular sizes, such as insulin, parathyroid hormone, interleukin 3, erythropoietin, albumin, transferrin, and Factor VIII to patients in need of these products

The Casing of the Conduit

The casing 75 of the conduit (CC) portion of the device is a material constructed of a biologically inert polymer. Through the conduit, fluids are directly delivered to or withdrawn from the blood stream if desired. In certain embodiments, the casing is manufactured in a manner which makes the material permeable. The CC then allows passage of diffusible substances into the outer chamber, which may be filled with a medium (e.g. gel or other diffusable medium) through which such substances diffuse, and subsequently diffuse across the OPJ into the host blood stream. Thus, the conduit can serve as a reservoir for the sustained release of drugs or biologics. In this specialized case, the transport properties of the casing wall can be tailored to control the rate of delivery of a particular agent into the outer chamber and across the OPJ and into the bloodstream.

In one mode of use, the central lumen contains a volume of medium comprising an agent which diffuses through the casing, the outer chamber and the outer jacket, of the device into the blood stream. When the concentration or activity of the agent in the lumen is sufficiently depleted, a syringe is attached to the access port, for replacing the spent medium with fresh medium and agent using techniques similar to withdrawing and replacing fluids in standard indwelling or tunneled catheters.

The casing also blocks living cells from entering the inner lumen and entering the bloodstream. More specifically, the casing of the conduit is produced in such a manner that it has transport characteristics that can be used to quicken or slow or sustain the delivery of nutrients, growth factors or drugs into the outer chamber to sustain or augment the viability of the sequestered cells or otherwise influence their biology in some advantageous way. For example, by augmenting oxygen delivery from a saturated oxygen-carrying artificial hemoglobin polymer or perflurocarbon, the contents of the inner casing can be used to maintain viability of the cells housed in the outer chamber.

In specialized cases, where the biological moiety may be immunologically incompatible, the permeability properties of the casing wall are matched to those of the OPJ, as known to those skilled in the art in making immunoisolatory bioartificial implant systems. In such cases where the cells are nonimmunoreactive as in the use of autologous stem cells and autografts derived cell lines where rejection is not a problem, the inner casing of the conduit may be freely permeable.

The materials used to form the casing of the conduit have similar properties and attributes as described for the outer permeable jacket portion of the device including, biocompatibility, and biostability, and therefore are chosen from the same types of materials as described for the OPJ. Preferred substances include polyurethanes and blends thereof or permeable silicone based elastomers.

The Chamber of the Device

The chamber 30 of the device is constructed to provide a suitable local environment to maintain the functional efficacy of the biological moiety. In some embodiments, the chamber comprises a liquid medium sufficient to maintain cell viability. Liquid cores are particularly suitable for maintaining transformed cells, such as PC12 cells. In other embodiments, the core comprises a gel matrix which immobilizes and distributes the cells, thereby reducing the formation of dense cellular agglomerations. The gel matrix may be composed of polyelectrolytes, temperature sensitive hydrogels, conventional or typical hydrogels or extracellular matrix components.

Other cells, particularly primary cells or tissues, tend to adhere to each other and form dense agglomerations which develop central necrotic regions due to the relative inaccessibility of nutrients and oxygen to cells embedded within the agglomerated masses. These dense cellular masses can form slowly, as a result of cell growth into dense colonies, or rapidly, upon the reassociation of freshly-dispersed cells or tissue mediated by cell-surface adhesion proteins. Cells or tissues which are highly metabolically active are particularly susceptible to the effects of oxygen or nutrient deprivation, and die shortly after becoming embedded in the center of an agglomerate. Many endocrine tissues, which normally are sustained by dense capillary beds, exhibit this behavior; islets of Langerhans and adrenal chromaffin cells are particularly sensitive. Cells or tissues which exhibit this behavior perform most satisfactorily in devices comprising a hydrogel matrix core, sufficient to immobilize the cells or tissue, thereby preserving the access of nutrients and oxygen to the majority of them.

Suitably, the medium disposed in the chamber may be composed of a matrix formed by a hydrogel which stabilizes the position of the cells in cell clumps. The term “hydrogel” herein refers to a three dimensional network of cross-linked hydrophilic polymers. The network is in the form of a gel substantially composed of water, preferably but not limited to gels being greater than 90% water. Cross-linked hydrogels can also be considered solids because they do not flow or deform without appreciable applied shear stress.

Compositions which form hydrogels fall into three classes for the purposes of this application. The first class are polyanions that carries a net negative charge and is typified by alginate. The second class carries a net positive charge and is typified by extracellular matrix components such as collagen and laminin. Examples of commercially available extracellular matrix components include Matrigel.™. and Vitrogen.™. The third class is net neutral in charge. An example of a net neutral hydrogel is highly crosslinked polyethylene oxide, or polyvinyalcohol.

Optionally, the medium contains substances which support or promote the function of the isolated cells. These substances include natural or synthetic nutrient sources, growth factors or growth regulatory substances, or a population of feeder or accessory cells or oxygen carriers such as hemoglobins and fluorocarbons.

Fibroblasts generally survive well in a positively charged matrix and are thus suitably enclosed in extracellular-matrix type hydrogels. Certain cell types tend to multiply rapidly and could overgrow the space available within the core if they do not exhibit growth arrest. If the isolated cells do not exhibit growth arrest upon confluence, substances which induce quiescence can be included in the interior of the device. In some instances, a hydrogel core may suffice to limit continued proliferation. For example, a hydrogel matrix precursor solution can be included but not exposed to polymerizing conditions. In the case of sodium alginate, a hydrogel will form slowly after implantation as calcium ions are scavenged from the surrounding tissues. Alternatively, growth inhibitory factors, or stimulators of differentiation can be incorporated into microbeads of a slowly degraded polymer such as polycarbonate, and cosuspended with the product-secreting cells. For instance, NGF or FGF can be used to stimulate PC-12 cell differentiation and terminate cell division.

In other circumstances, the immobilizing hydrogel matrix further performs the additional function of producing or preserving functional units of a size and/or shape appropriate for maintaining desirable characteristics of the isolated cells. Moreover, the presence of the matrix allows maintenance of a uniform distribution of cells or clusters of cells within the chamber device (i.e., the matrix prevents settling and decreases mobility of the included cells).

Primary cells or tissues may be useful with the device of the instant invention for various medical applications. For regulatory reasons and reasons of patient safety, it may sometimes be useful to employ as sources for primary cultures, animals of carefully controlled hereditary and developmental background. The presence of unwanted virus, bacteria and other pathogens may be limited through the use of specific pathogen free or gnotobiotic source animals. References and methods for the establishment, care and use of specific pathogen free and gnotobiotic herds are provided by Maniatis, O. P., et al. Can. J. Med., 42, p. 428 (1978); Matthews P. J., et al., Recent Advances in Germ Free Research, pp. 61-64, Tokai Univ. Press (1981), and in the National Accreditation Standards publication of the National SPF Swine Accrediting Agency, Inc., Conrad, Iowa.

Optionally, a matrix filled chamber can also contain materials which support or promote the function of the isolated cells. For example, extracellular matrix (ECM) components can be included to promote specific attachment or adhesion of the isolated cells. A combination of ECM components which is particularly suitable for fostering the growth of certain types of cells is taught in Kleinman et al., U.S. Pat. No. 4,829,000. The matrix can provide a reservoir for soluble or releasable substances, such as growth factors or growth regulatory substances, or for natural or synthetic substances which enhance or improve the supply of nutrients or oxygen to the isolated cells. Thus, it can function in a manner similar to the bone marrow ECM, which has been reported to behave as a slow-release reservoir for myeloid lineage-specific growth factors such as granulocyte-macrophage colony stimulating factor (gmcsf). Gordon, M. Y., et al., Nature, 326, pp. 403-405 (1987). Thus, the matrix can function as a reservoir for growth factors (e.g., prolactin, or insulin-like growth factor 2), growth regulatory substances such as transforming growth factor .beta. (TGF.beta.) or the retinoblastoma gene protein or nutrient-transport enhancers (e.g., perfluorocarbons, which can enhance the concentration of dissolved oxygen in the core). Certain of these substances are also appropriate for inclusion in liquid media.

Additionally, a population of feeder or accessory cells can be co-isolated within the chamber. For example, hepatocytes can be co-isolated with endothelial accessory cells, Cai, Z., et al., Artificial Organs, 12, pp. 388-393 (1988), or mixed with islet cells, Ricordi C., et al., Transplantation 45, pp. 1148-1151 (1987), or adrenal chromaffin cells can be co-isolated with accessory cells which provide nerve growth factor (NGF), a substance needed by the chromaffin cells. In the latter case, fibroblasts which have been transfected with an expression vector for NGF can be used as accessory cells.

Load the Device—Loading Density

Four important factors influencing loading density, i.e. the number of cells or amount of tissue placed within the chamber of the device are: (1) device size and geometry; (2) mitotic activity within the chamber; (3) viscosity requirements for core preparation and or loading; and (4) pre-implantation assay and qualification requirements.

With respect to the first of these factors, (device size and geometry), the diffusion of critical nutrients and metabolic requirements into the cells as well as diffusion of waste products away from the cells are critical to the continued viability of the cells. Since diffusional access to the contents of the chamber is limited by outer membrane surface area, surface to volume relationships of various shapes and size chambers will be critical in determining how much viable tissue can be maintained within the chamber.

Among the metabolic requirements met by diffusion of substances into the device is the requirement for oxygen. The oxygen requirements of the specific cells must be determined for the cell of choice. Methods and references for determination of oxygen metabolism are given in Wilson D. F. et al., J. Biol. Chem., 263, pp. 2712-2718, (1988). The oxygen requirement for islet cells has been applied to coupled diffusion reaction models accounting for diffusional transport from surrounding tissue through the device wall and tissue compartment (core), and used to calculate the expected viability of islet cells in a number of bioimplant devices of different sizes and configurations, after the method of Dionne, K. E., Ph.D. Thesis, Massachusetts Institute of Technology (1989). For intact pancreatic islets, these calculations agree well with experimental observations.

Optimal cell volumes may be similarly calculated for the geometries of the device of the invention. Actual loading densities will consider not only these diffusional considerations but also the other considerations given below.

With respect to the second factor (cell division), if the cells selected are expected to be actively dividing while in the device, then they will continue to divide until they fill the available space, or until phenomena such as contact inhibition limit further division. For replicating cells, the geometry and size of the device will be chosen so that complete filling of the chamber will not lead to deprivation of critical nutrients due to diffusional limitations.

In general, for cells not expected to divide within the device, such as chromaffin cells, pancreatic islet cells and the like, the appropriate cell densities will be calculated from the diffusional considerations listed.

With respect to the third factor (viscosity of core materials) cells in densities occupying up to 70% of the chamber volume can be viable, but cell solutions in this concentration range would have considerable viscosity. Introduction of cells in a very viscous solution into the chamber could be prohibitively difficult.

Finally, with respect to the fourth factor (preimplantation and assay requirements), in many cases, a certain amount of time will be required between device preparation and implantation. For instance, it may be important to qualify the device in terms of its biological activity. Thus, in the case of mitotically active cells, preferred loading density will also consider the number of cells which must be present in order to perform the qualification assay.

Establishing Efficacy of Biologically Active Moiety

In most cases, prior to implantation in vivo it will be important to use in vitro assays to establish the efficacy of the biologically active moiety within the chamber. Devices containing the moiety of interest can be constructed and analyzed using model systems. In a preferred embodiment of the instant invention, the diffusion of glucose into the chamber is used to stimulate insulin release from pancreatic islet cells. The appearance of insulin outside the chamber is monitored through the use of an appropriately specific assay. Such procedures allow the determination of the efficacy of the device on a per cell or unit volume basis.

Following the above guidelines for device loading and for determination of device efficacy, the actual device size for implantation will then be determined by the amount of biological activity required for the particular application. In the case of secretory cells releasing therapeutic substances, standard dosage considerations and criteria known to the art will be used to determine the amount of secretory substance required. Factors to be considered include; the size and weight of the recipient; the productivity or functional level of the cells; and, where appropriate, the normal productivity or metabolic activity of the organ or tissue whose function is being replaced or augmented. It is also important to consider that a fraction of the cells may not survive the immunoisolation and implantation procedures, as well as whether the recipient has a preexisting condition which can interfere with the efficacy of the implant. Devices of the instant invention can easily be manufactured which contain many thousands of cells. In preferred embodiments, therapeutically useful immunoisolatory devices used to provide insulin to insulin deficient rats contained on the order of 1,000 islets. Larger devices can also be conveniently prepared by the method of the current invention.

Chamber Comprising Non-Cellular Biotherapeutics

The device can also be used as a reservoir for the controlled delivery of needed drugs or biotherapeutics. In such cases, the chamber, rather than containing cells or tissues, contains a suitable concentration of the selected drug or biotherapeutic, i.e. a biologically active molecule or substance such as an enzyme, trophic factor, hormone, or biological response modifier. The present invention applies to the administration of beneficial agents in general, which include any physiologically or pharmacologically active substance. The beneficial agent may be any of the agents which are known to be delivered to the body of a human or an animal such as drug agents, medicaments, vitamins, nutrients, or the like.

Method of Delivery: Therapeutic Methods using the Device:

The implant device of the invention is used for individuals who require therapeutic treatment which requires administration or delivery of a biological moiety to achieve a therapeutic effect. The device comprising biological moieties of interest is implanted into an individual's central venous vasculature for a sufficient period of time to delivery a sufficient amount of the biological moiety to the individual to achieve a therapeutic effect.

Treatment of Type I Diabetes (Delivery of a Product Secreted from Biological Moiety):

Practical application of the device for treatment type I diabetes involves fabrication of the device as described and sterilization by standard techniques. Pancreatic tissue is harvested using standard techniques and Islets of Langerhans isolated and combined with an immobilization matrix including but not limited to sodium alginate. The slurry is injected into the chamber.

The sterile-cell containing device and port is be implanted into any one of the number of peripheral veins including but not limited to the sites used for typical placement of an indwelling peripherally inserted central venous catheter using sterile methods well known to those in the field. Removal is achieved through standard surgical methods.

Products which can be delivered using the instant device include a wide variety of factors normally secreted by various organs or tissues. For example, insulin can be delivered to a diabetic patient or Factor VIII to a Type A hemophiliac.

Other products which can be delivered through use of the instant device include trophic factors such as erythropoietin, growth hormone, Substance P, and neurotensin. This invention is useful for treating individuals suffering from acute and/or chronic pain, by delivery of an analgesic or pain reducing substance to the individual. Such pain reducing substances include enkephalins, catecholamines and other opioid peptides. Such compounds may be secreted by, e.g., adrenal chromaffin cells. Another family of products suited to delivery by the instant device comprises biological response modifiers, including lymphokines and cytokines. Antibodies from antibody secreting cells may also be delivered. Specific antibodies which may be useful include those towards tumor specific antigens. The release of antibodies may also be useful in decreasing circulating levels of compounds such as hormones or growth factors. These products are useful in the treatment of a wide variety of diseases, inflammatory conditions or disorders, and cancers.

The instant device can also be used to restore or augment vital metabolic functions, such as the removal of toxins or harmful metabolites (e.g., cholesterol) from the bloodstream by cells such as hepatocytes. The method and device of the instant invention make possible the implantation of cells without the concomitant need to immunosuppress the recipient for the duration of treatment. Through use of the biocompatible immunoisolatory device, homeostasis of particular substances can be restored and maintained for extended periods of time. The instant device may contain a multiplicity of cells, such that implantation of a single device can be sufficient to provide an effective amount of the needed substances or functions to treat multiple ailments simultaneously in an individual.

Many uses of cellular implants to treat diseases have been proposed, including treatment of hemophilia, dwarfism, anemia, kidney failure, anemia, chronic pain, Alzheimer's disease, fulminant hepatic failure, etc.

A wide variety of biologically active moieties or cells may be used in this invention. These include well known, publicly available immortalized cell lines as well as primary cell cultures. Examples of publicly available cell lines suitable for the practice of this invention include, baby hamster kidney (BHK), chinese hamster ovary (CHO), mouse fibroblast (L-M), NIH Swiss mouse embryo (NIH/3T3), African green monkey cell lines (including COS-a, COS-7, BSC-1, BSC-40, BMT-10 and Vero), rat adrenal pheochromocytoma (PC12) and rat glial tumor (C6). Primary cells that may be used according to the present invention include, bFGF-responsive neural progenitor-stem cells derived from the CNS of mammals (Richards et al., Proc. Natl. Acad. Sci. USA 89, pp. 8591-8595 (1992); Ray et al., Proc. Natl. Acad. Sci. USA, 90, pp. 3602-3606 (1993)), primary fibroblasts, Schwann cells, astrocytes, .beta.-TC cells, Hep-G2 cells, AT T20 cells, oligodendrocytes and their precursors, myoblasts, adrenal chromaffin cells, and the like.

The choice of biologically active moiety or cell depends upon the intended application. The encapsulated cells may be chosen for secretion of a neurotransmitter. Such neurotransmitters include dopamine, gamma aminobutyric acid (GABA), serotonin, acetylcholine, noradrenaline, epinephrine, glutamic acid, and other peptide neurotransmitters. Cells can also be employed which synthesize and secrete agonists, analogs, derivatives or fragments of neurotransmitters which are active, including, for example, cells which secrete bromocriptine, a dopamine agonist, and cells which secrete anti-tumor agents such as anti-angiogenesis factors.

The encapsulated cells can also be chosen for their secretion of hormones, cytokines, growth factors, trophic factors, angiogensis factors, antibodies, blood coagulation factors, lymphokines, enzymes, and other therapeutic agents or agonists, precursors, active analogs, or active fragments thereof. These include enkephalins, catecholamines, endorphins, dynorphin, insulin, factor VIII, erythropoietin, Substance P, nerve growth factor (NGF), Glial derived Neurotrophic Factor (GNDF), platelet-derived growth factor (PDGF), epidermal growth factor (EGF), brain-derived neurotrophic factor (BDNF), neurotrophin-3 (NT-3), an array of fibroblast growth factors, and ciliary neurotrophic factor.

Alternatively, one or more biologically active molecules may be delivered into the capsule. For example, the capsule may contain one or more cells or substances which “scavenge” cholesterol, or other biological factors, from the host.

Techniques and procedures for isolating cells or tissues which produce a selected product are known to those skilled in the art, or can be adapted from known procedures with no more than routine experimentation. For example, islets of Langerhans can be isolated from a large-animal pancreas (e.g., human or porcine) using a combination of mechanical distention and collagenase digestion, as described by Scharp, D. W., et al., U.S. Pat. No. 4,868,121. Islets may be isolated from small animals such as rats by the method of Scharp, et al., Diabetes 29, suppl. 1, pp. 19-30 (1980). Similarly, hepatocytes can be isolated from liver tissue using collagenase digestion followed by tissue fractionation, as described by Sun, A. M., et al., Biomat., Art. Cells, Art. Org., 15, pp. 483-496 (1987). Adrenal Chromaffin cells may be isolated by the method of Livett, B. G., Physiology Reviews, 64, pp. 1103-1161 (1984).

Many cellular products which are difficult to provide using primary donor tissues can be provided using immortalized cells or cell lines and an ex vivo approach. Immortalized cells are those which are capable of indefinite replication but which exhibit contact inhibition upon confluence and are not tumorigenic. An example of an immortalized cell line is the rat pheochromocytoma cell line PC12. Transformed cells or cell lines can be used in a similar manner. Transformed cells are unlike merely immortalized cells in that they do not exhibit contact inhibition upon confluence, and form tumors when implanted into an allogeneic host. Immortalization can allow the use of rare or notoriously fragile cell or tissue types for the long-term delivery of a chosen product or metabolic function. Suitable techniques for the immortalization of cells are described in Land H. et al., Nature 304, pp. 596-602 (1983) and Cepko, C. L., Neuron 1, pp. 345-353 (1988). Candidate cell lines include genetically engineered beta-cell lines which secrete insulin such as NIT cells (Hamaguchi, K., et al., Diabetes 40, p. 842 (1991)), RIN cells (Chick, W. L., et al., Proc. Natl. Acad. Sci. USA, 74, pp. 628-632 (1977)), ATT cells (Hughes, S. D., et al, Proc. Natl. Acad. Sci. USA, 89, pp. 688-692 (1992)), CHO cells (Matsumoto, M., et al, 1990, Proc. Natl. Acad. Sci. USA, 87, pp. 9133-9137 (1990)), and beta-TC-3 cells (Tal, M., et al, 1992, Mol. Cell Biol., 12, pp. 422-432 (1992)). Additionally, recombinant cells or cell lines can be engineered to provide novel products or functions and combinations thereof, using a wide variety of techniques well known to those of ordinary skill in the art.

For example, fibroblasts can be transfected with an expression vector for the chosen product (e.g., nerve growth factor, erythropoietin, insulin, or Factor VIII). It should be recognized however, that expression of a recombinant protein in a cell type which does not normally express the protein may lead to unregulated expression which may not be desirable for certain medical applications.

B-cell hybridomas secreting a selected monoclonal antibody, or T-cell hybridomas secreting a selected lymphokine, can also be used. It may be particularly desirable to deliver a monoclonal antibody or fraction thereof, which neutralizes the biological activity of a disregulated biological response modifier using the instant invention. Engineered cells which secrete soluble fragments of receptors for these biological response modifiers can be used in a similar fashion. The disregulation or overproduction of particular biological response modifiers has been implicated in the etiology of certain cancers.

The encapsulated material can be tissue or cells able to secrete such antinociceptive agents, including any one of catecholamines, enkephalins, opioid peptides or mixtures thereof. Preferably catecholamines are secreted, most preferably a mixture of catecholamines and enkephalins. Typically, the encapsulated material can be tissue of the adrenal medulla, or more particularly, adrenal medulla chromaffin cells. Additionally, genetically engineered cell lines or other naturally occurring cell lines able to secrete at least one pain reducing agent such as a catecholamine, enkephalin, opioid peptide, or agonist analog thereof, can be used.

If the cells are replicating cells or cell lines adapted to growth in vitro, it is particularly advantageous to generate a cell bank of these cells. A particular advantage of a cell bank is that it is a source of cells prepared from the same culture or batch of cells. That is, all cells originated from the same source of cells and have been exposed to the same conditions and stresses.

Therefore, the vials can be treated as identical clones. In the transplantation context, this greatly facilitates the production of identical or replacement devices. It also allows simplified testing protocols which assure that implanted cells are free of retroviruses and the like. It may also allow for parallel monitoring of devices in vivo and in vitro, thus allowing investigation of effects or factors unique to residence in vivo.

Method of Implanting the Device

First, the device is loaded with biological moiety (ies). Placement technique for the loaded device is the same as for PICCs and CVCs, and is typically performed by a radiologist using standard imaging techniques. The standard approach uses a 22G angiocatheter (Johnson & Johnson Medical, Arlington Tex.). Once the angiocath is in the vein, an 0.018″ guidewire is advanced. The preferred placement technique uses ultrasound guidance. Standard technique is followed using a peel away introducer. Tunneled central lines which may contain a Dacron cuff that becomes incorporated into the subcutaneous tissue are introduced through the puncture site and are connected to the access port and are considered permanent venous access devices. These are more costly to place that those anchored by external adhesive, but are the most practical choice for the long-term therapy of the present invention.

Methods of Manufacture

The present invention is directed to the fabrication of devices which enclose biologically active moieties, which are completely biocompatible over extended periods of time.

The instant invention thus relates to a method for making a device which is inserted in the bloodstream, modifies the extracellular fluid microenvironment for a period of time using a variety of technologies including transplanted cells, bioartificial organs and sustained delivery devices such as polymeric implants, and maintains an opening in a biological channel such as a blood vessel which can be used to put fluid in and take fluid out.

Method of Isolating Cells

This invention also pertains to a method sequestering biologically active moieties within the device from the host, i.e. of isolating or protecting biologically active moieties, such as implanted cells, tissues, or other materials from immunological attack. The methods and devices of the instant invention are useful to deliver a wide range of cellular products, including high molecular weight products, to an individual in need of them, and/or to provide needed metabolic functions to an individual, such as the removal of harmful substances.

### EXAMPLES

Example of Device for Treatment of Type I Diabetes

Type 1 diabetes, also called juvenile diabetes or insulin-dependent diabetes, is usually first diagnosed in children, teenagers, or young adults. In this form of diabetes, the islet cells of the pancreas no longer make insulin because the body's immune system has attacked and destroyed them.

Although Type I diabetes can be controlled with insulin injections, this treatment is not a cure. Safe blood glucose levels cannot be precisely and continuously maintained. An overdose of insulin results in hypoglycemia, a low level of blood glucose. This can lead to confusion, loss of consciousness, coma, and even death. On the other hand, extended high blood glucose levels result in significant long-term complications, such as damage or failure of various bodily organs including the eyes, kidneys, nerves, heart and blood vessels.

A cell-based therapy for Type I diabetes would restore a patient's ability to control blood glucose levels continuously and accurately, without the need for insulin injections or blood glucose monitoring. The transplanted cells would automatically respond to changing blood glucose levels just as the patient's original islet cells.

The present invention may be used to deliver islets from various sources, including humans, animals, and genetically manipulated cell lines, into a human Type I diabetic patient. In the following example, the transplantation of porcine islets into a human diabetic will be described.

The device will be sized based on the following assumptions: 1) adult dosage=2,000 IEQ/kg body weight, 2) adult body weight=70 kg, an IEQ is a 150 um diameter islet cell, and 3) the islet suspension concentration will be approximately 25%. These assumptions result in the following requirements for the device: the device must accommodate approximately a 1 ml suspension of 140,000 IEQ.

The dimensions of the device will be similar to a 6 french catheter. It will have an OD of 0.206 cm and have a central lumen (D=0.046 cm) for guidewire insertion. In order to meet the minimum internal volume requirement of 1 ml, the device will be approximately 35 cm in length.

Porcine islets will be obtained using standard islet isolation techniques, which include surgical pancreas procurement, enzymatic tissue digestion, islet purification, and islet culturing (U.S. Pat. Nos. 6,303,355; 6,365,385, incorporated by reference). The islets will then be suspended in a buffered 1-2% sodium alginate solution.

Standard extrusion fabrication technologies which are known in the art are used in making the device (e.g. Engineered Materials Handbook, Desk edition, 1995, ASM International; Engineered Materials Handbook Vol 3: Adhesives and Sealants, 1990, ASM International; Plastics Extrusion Technology Handbook, 2n edition, S Levy and J F Carley, 1989 Industrial Press Inc.).

A method of making a device for use in a diabetic adult patient for insertion and sustained residence in the blood stream and for delivery to the bloodstream of pancreatic islets or glucose responsive insulin secreting cell lines involves the steps of: fabricating the outer permeable jacket by dry, jet wet spinning phase inversion methods or particulate leaching after standard catheter extrusion, with a wall varying in size from 20-500 microns but not limited to this range and an outer diameter of 0.206 cm. The casing of the conduit is formed by standard catheter extrusion technology as known to those skilled in the art of intravascular catheter manufacture with a wall varying in size from 20-500 microns but not limited to this range and an inner diameter of 0.046 cm. The outer jacket is connected to the inner conduit by the supporting internal structure using standard adhesive technology or melt adhesion to form an integrated structure as shown in FIGS. 3-6. The outer chamber is then sealed at the distal end by adhesive such as a UV cured Luxtrak (Ablestik labs). The proximal end is connected to tubing attached to a syringe pump using the inner lumen as a guide and female connector. The outer wall of the OPJ is wetted by ultrafiltrating a 0.4 micron filtered and degassed 20% ethanol solution through the outer permeable jacket. The ethanol solution is replaced with sterile 0.4 micron filtered water which is then infused though the lumen and across the OPJ to remove all traces of the ethanol solution. A 2% solution of alginate in low calcium containing physiological salt solution mixed with pancreatic islets, in this instance the biological moieties 55, is then infused via the syringe pump in a predetermined volume into the outer permeable chamber. The proximal tubing is disconnected and the proximal end of the cell containing portion of the device is sealed by a tubular silicone connector that is anchored with adhesive known to those skilled in the art of intravascular catheter manufacture. The entire structure is then connected to the access port as is routine in clinical practice. The implantable access port is made of a composite structure including a body molded of metal or metallic to allow and at least one surface facing the skin that is made of a resealable and flexible elastomer that can be pierced or accessed through the skin with standard stainless steel hypodermic needles. The access port is manufactured using standard methods known to those skilled in the art such as described for the manufacture of Infusaport, or Port-A-Cath and implanted using standard clinical approaches for tunneled central venous catheter lines.

Intraperitoneal and Subcutaneous Placement of the Device

The device of the invention is suitable for implanatation into the peritoneal cavity of the host, as well as subcutaneous implantation. The implant device of the invention is used for individuals who require therapeutic treatment which requires administration or delivery of a biological moiety to achieve a therapeutic effect. The device comprising biological moieties of interest is implanted into an individual's peritoneal cavity or subcutaneously for a sufficient period of time to delivery a sufficient amount of the biological moiety to the individual to achieve a therapeutic effect. Methods of placing bioartificial implants intraperiotoneally or subcutaneously are well known in the art for delivering secreted product or metabolic function to a host (U.S. Pat. Nos. 5,916,554, and 6,372,244, both incorporated by reference).

1. An device for implanting biological moieties in a host, comprising:
a. a tube which comprises proximal and distal ends, at least a portion of the length of said tube bound by an isolating jacket;
b. a conduit bound by a casing which defines a lumen therein, said conduit mounted between the proximal and distal ends of said tube; and
c. a chamber within said tube defined by said jacket and said casing.

2. The device of claim 1 wherein said jacket comprises an elastomer selected from the group consisting of silicon, polyurethane, or a blends thereof.

3. The device of claim 1 wherein the casing comprises semi-permeable material selected from the group consisting of silicon, polyurethane, or a blends thereof.

4. The device of claim 1 wherein the surface of the jacket in contact with the blood stream is biocompatible.

5. The device of claim 4 wherein the surface of the jacket comprises anti-thrombotic material.

6. The device of claim 1 wherein medium is disposed in said chamber, said lumen, or said chamber and lumen.

7. The device of claim 6 wherein said medium comprises solutes comprising biological moieties.

8. The device of claim 6 wherein solutes diffusively transport between said lumen, said chamber, and said bloodstream.

9. The device of claim 1 wherein said lumen is sized for over-the-wire insertion in a blood vessel.

10. The device of claim 1 wherein one or both ends of the lumen is fitted with either a seal or a valve.

11. The device of claim 1 wherein said conduit is sufficiently rigid to resist kinking of the device upon insertion into and travel in a peripheral blood vessel.

12. The device of claim 1 wherein said chamber comprises at least one support member positioned between said conduit and said semi-permeable jacket and disposed for engaging an interior surface of the jacket.

13. The device of claim 12 wherein said support member is helical.

14. The device of claim 13 wherein said support member comprises a helical shaft and a bore formed the length of the shaft.

15. The device of claim 1 wherein a catheter having a proximal end and a distal end is mounted in fluid communication on the conduit disposed at the proximal end of the device.

16. The device of claim 15 wherein an injection port is mounted on the proximal end of said catheter in fluid communication with the lumen.

17. The device of claim 16 wherein said injection port is implantable.

18. A device for implanting biological moieties in a host, comprising:
a. a tube which comprises proximal and distal ends, at least a portion of the length of said tube bound by an isolating jacket;
b. a conduit bound by a casing which defines a lumen therein, said conduit mounted between the proximal and distal ends of said tube;
c. a chamber within said tube defined by said jacket and said casing;
d. at least one support member positioned between said conduit and said jacket and disposed for engaging an interior surface of the jacket.

19. The device of claim 18 wherein a catheter having a proximal end and a distal end is mounted in fluid communication on the conduit disposed at the proximal end of the device.

20. The device of claim 19 wherein an injection port is mounted on the proximal end of said catheter in fluid communication with the lumen.

21. The device of claim 20 wherein said injection port is implantable.

22. The device of claim 18 wherein said jacket comprises an elastomer selected from the group consisting of silicon, polyurethane, or a blends thereof.

23. The device of claim 18 wherein the casing comprises semi-permeable material selected from the group consisting of silicon, polyurethane, or a blends thereof.

24. The device of claim 18 wherein the surface of the jacket in contact with the blood stream is biocompatible.

25. The device of claim 24 wherein the surface of the jacket comprises anti-thrombotic material.

26. The device of claim 18 wherein medium is disposed in said chamber, said lumen, or said chamber and lumen.

27. The device of claim 26 wherein said medium comprises solutes comprising biological moieties.

28. The device of claim 26 wherein solutes diffusively transport between said lumen, said chamber, and said bloodstream.

29. The device of claim 18 wherein said lumen is sized for over-the-wire insertion in a blood vessel.

30. The device of claim 18 wherein one or both ends of the lumen is fitted with either a seal or a valve.

31. The device of claim 18 wherein said conduit is sufficiently rigid to resist kinking of the device upon insertion into and travel in a peripheral blood vessel.

32. The device of claim 18 wherein said support member is helical and comprises a helical shaft and a bore formed the length of the shaft.

33. A method of treating an individual in need of therapeutic treatment which involves administration of a biological moiety, the method comprising the step of introducing the device of claim 1 into the central venous vasculature for a sufficient period to deliver a sufficient amount of said biological moiety to the individual to achieve a therapeutic effect.

34. The method of claim 33 wherein said individual requires therapeutic treatment for diabetes.

35. A method of introducing a biological implant into an individual, comprising the step of inserting the device of claim 1 into the central venous vasculature.

36. The method of claim 35 wherein said individual requires therapeutic treatment for diabetes.

37. A method of treating an individual in need of therapeutic treatment which involves administration of a biological moiety, the method comprising the step of introducing the device of claim 1 into the peritoneal cavity or subcutaneous tissue for a sufficient period to deliver a sufficient amount of said biological moiety to the individual to achieve a therapeutic effect.
